(19) United States
(12) Patent Application Publication
(10) Pub. No.: US 2023/0339877 A1
(43) Pub. Date: Oct. 26, 2023

(54) Synthesis and Composition of Neurolenin D Mesylate and Related Compounds - New Drugs for the Treatment of Parasitic Nematode Infections in Humans, Animals and Plants

(71) Applicant: Smith College, Northampton, MA (US)

(72) Inventors: Kristine Trotta, San Francisco, CA (US); Lydia DeAngelo, Cleveland, OH (US); Susan J. Haynes, Ashfield, MA (US); Catherine McGeough, Boston, MA (US); Monalisa Munia, Northampton, MA (US); Peyton Higgins, Stoughton, WI (US); Steven A. Williams, North Hatfield, MA (US); Kevin Shea, Longmeadow, MA (US)

(21) Appl. No.: 18/304,411

(22) Filed: Apr. 21, 2023

Related U.S. Application Data
(60) Provisional application No. 63/333,826, filed on Apr. 22, 2022.

(51) Int. Cl. C07D 307/93; A61P 33/10

(52) U.S. Cl. CPC C07D 307/93; A61P 33/10

(57) ABSTRACT

The natural product neurolenin D is isolated from Neurolaena lobata using continuous extraction, stirring over charcoal, flash column chromatography, and recrystallization. The secondary alcohol in this molecule is converted into a methane sulfonyl ester (mesylate) to generate a new molecule, neurolenin D mesylate. This previously unknown molecule has been tested for biological activity in assays for human toxicity, mutagenicity, and nematocidal potency. It is not toxic or mutagenic and demonstrates the ability to kill nematodes responsible for the neglected tropical disease lymphatic filariasis and is likely to have the same ability to kill other nematodes responsible for a wide range of diseases in humans, animals and plants. Additional esters of neurolenin D are claimed and procedures are outlined for their synthesis.

### TECHNICAL FIELD

The present invention relates to the development of improved drugs for the treatment of infections caused by parasitic nematodes.

### CROSS-REFERENCE TO RELATED APPLICATIONS

This patent application claims the benefit of U.S. Provisional Pat. Application No. 63/333,826, filed Apr. 22, 2022, which application is hereby incorporated, in its entirety, by reference.

### BACKGROUND ART

Parasitic nematodes infect up to 1.5 billion people worldwide and untold billions of economically valuable plants and animals. Lymphatic filarisis (LF) is a neglected tropical disease that caused by the parasitic nematodes Wuchereria bancrofti, Brugia malayi, and Brugia timori. LF is the second leading cause of long-term disability worldwide. Because the drugs used to treat diseases caused by parasitic nematodes have been used routinely for many years and have been overused, drug resistance is a well-documented problem in some species and drives the need for new and potent drugs to kill these common pathogens. Another concern with current drugs is that they typically do not kill adult parasites. The result is that the drugs must be used repeatedly to prevent transmission of the infection. Another concern with current treatments is that caution must be exercised not to kill the parasites in an infected individual too quickly since this can cause a potentially dangerous immunological reaction. Patients with the filarial parasite Loa loa have died when the parasites were killed too quickly.

### SUMMARY OF THE EMBODIMENTS

Neurolenin D mesylate, a novel compound, having the structural formula

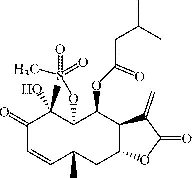 [SMILES: C=C1C(=O)O[C@@H]2C[C@H](C)/C=C\C(=O)[C@](C)(O)[C@@H](OS(C)(=O)=O)[C@@H](OC(=O)CC(C)C)[C@@H]12]

is herein disclosed.

In some embodiments, formulations of neurolenin D mesylate are provided for the treatment of an organism suffering from a disease caused by an infection with a parasitic nematode, the organism selected from the group consisting of human, animal, and plant.

A method of synthesizing neurolenin D mesylate is disclosed, according to which neurolenin D is reacted with mesyl chloride to form Neurolenin D mesylate.

In some embodiments, methods are disclosed for treating an organism suffering from a disease caused by an infection with a parasitic nematode, the organism selected from the group consisting of human, animal, and plant, the method comprising administering to the organism a therapeutically effective dose of neurolenin D mesylate. In some embodiments, the organism is a human. In some embodiments, the organism is a companion animal. In some embodiments, the organism is livestock. In some embodiments the organism is a plant.

In some embodiments the parasitic nematode is a causative agent of lymphatic filariasis and elephantiasis in humans. In some embodiments, the parasitic nematode is a causative agent of heartworm in dogs. In some embodiments, the parasitic nematode is a causative agent of a roundworm disease in livestock.

Herein disclosed are ten neurolenin D esters:

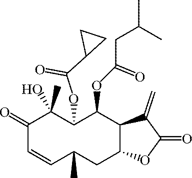 [SMILES: C=C1C(=O)O[C@@H]2C[C@H](C)/C=C\C(=O)[C@](C)(O)[C@@H](OC(=O)C3CC3)[C@@H](OC(=O)CC(C)C)[C@@H]12]

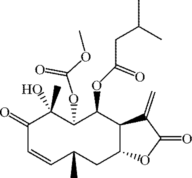 [SMILES: C=C1C(=O)O[C@@H]2C[C@H](C)/C=C\C(=O)[C@](C)(O)[C@@H](OC(=O)OC)[C@@H](OC(=O)CC(C)C)[C@@H]12]

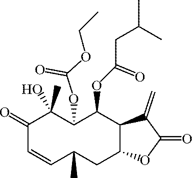 [SMILES: C=C1C(=O)O[C@@H]2C[C@H](C)/C=C\C(=O)[C@](C)(O)[C@@H](OC(=O)OCC)[C@@H](OC(=O)CC(C)C)[C@@H]12]

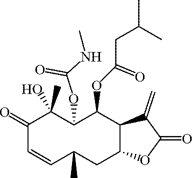 [SMILES: C=C1C(=O)O[C@@H]2C[C@H](C)/C=C\C(=O)[C@](C)(O)[C@@H](OC(=O)NC)[C@@H](OC(=O)CC(C)C)[C@@H]12]

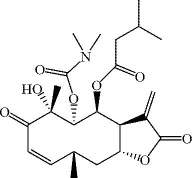 [SMILES: C=C1C(=O)O[C@@H]2C[C@H](C)/C=C\C(=O)[C@](C)(O)[C@@H](OC(=O)N(C)C)[C@@H](OC(=O)CC(C)C)[C@@H]12]

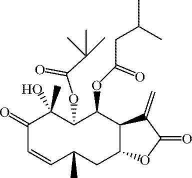 [SMILES: C=C1C(=O)O[C@@H]2C[C@H](C)/C=C\C(=O)[C@](C)(O)[C@@H](OC(=O)C(C)(C)C)[C@@H](OC(=O)CC(C)C)[C@@H]12]

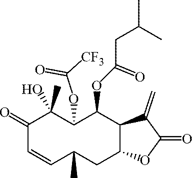 [SMILES: C=C1C(=O)O[C@@H]2C[C@H](C)/C=C\C(=O)[C@](C)(O)[C@@H](OC(=O)C(F)(F)F)[C@@H](OC(=O)CC(C)C)[C@@H]12]

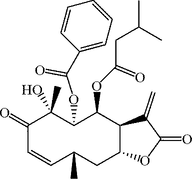 [SMILES: C=C1C(=O)O[C@@H]2C[C@H](C)/C=C\C(=O)[C@](C)(O)[C@@H](OC(=O)c3ccccc3)[C@@H](OC(=O)CC(C)C)[C@@H]12]

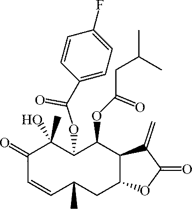 [SMILES: C=C1C(=O)O[C@@H]2C[C@H](C)/C=C\C(=O)[C@](C)(O)[C@@H](OC(=O)c3ccc(F)cc3)[C@@H](OC(=O)CC(C)C)[C@@H]12]

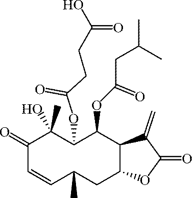 [SMILES: C=C1C(=O)O[C@@H]2C[C@H](C)/C=C\C(=O)[C@](C)(O)[C@@H](OC(=O)CCC(=O)O)[C@@H](OC(=O)CC(C)C)[C@@H]12]

In some embodiments, formulations of one or more of these ten neurolenin D esters are provided for the treatment of an organism suffering from a disease caused by a parasitic nematode, the organism selected from the group consisting of human, animal, and plant.

A method of synthesizing neurolenin D esters is disclosed, which includes a step of reacting neurolenin D with a compound having a formula selected from the group consisting of

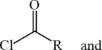

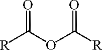

under reaction conditions which include the presence of an amine base. For these compounds, R is selected from the group consisting of:

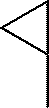

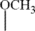

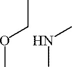

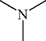

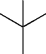

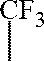

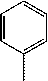

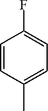

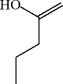

For some such embodiments the compound reacting with neurolenin D has the formula

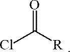

For other embodiments the compound reacting with neurolenin D has the formula

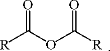

For some embodiments, the amine base is selected from the group consisting of pyridine, Diazabicycloundecene (DBU), imidazole, 4-Dimethylaminopyridine (DMAP), and 1,4-Diazabicyclo[2.2.2]octane (DABCO).

A method is herein disclosed of an organism suffering from a disease caused by an infection with a parasitic nematode, the organism selected from the group consisting of human, animal, and plant, the method comprising administering a therapeutically effective dose of one or more of the referenced ten neurolenin D esters. In some embodiments the organism is a human, a companion animal, livestock, or a plant.

In some embodiments the parasitic nematode causing the treated disease is selected from the group consisting of lymphatic filariasis, elephantiasis, and river-blindness in humans. In some embodiments the parasitic nematode is a causative agent of heartworm in dogs. In some embodiments the parasitic nematode is a causative agent of a roundworm disease in livestock.

### BRIEF DESCRIPTION OF THE DRAWINGS

The foregoing features of embodiments will be more readily understood by reference to the following detailed description, taken with reference to the accompanying drawings, in which:

FIG. 1 shows data demonstrating the ability of neurolenin D mesylate (NDM) to kill adult female Brugia pahangi parasites (BP AF) in culture.

FIG. 2 shows data demonstrating the ability of NDM to kill adult male Brugia pahangi parasites (BP AM) in culture.

### DETAILED DESCRIPTION OF SPECIFIC EMBODIMENTS

Definitions. As used in this description and the accompanying claims, the following terms shall have the meanings indicated, unless the context otherwise requires:

“Neurolenin D” is the chemical compound having the formula:

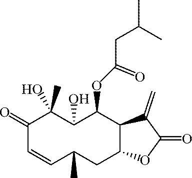 [SMILES: C=C1C(=O)O[C@@H]2C[C@H](C)/C=C\C(=O)[C@](C)(O)[C@@H](O)[C@@H](OC(=O)CC(C)C)[C@@H]12]

A method of purifying Neurolenin D from the plant Neurolaena lobata is described in Example 1 below.

“Neurolenin D Mesylate” (NDM) is the chemical compound having the formula:

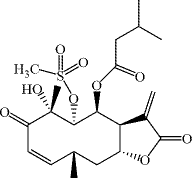 [SMILES: C=C1C(=O)O[C@@H]2C[C@H](C)/C=C\C(=O)[C@](C)(O)[C@@H](OS(C)(=O)=O)[C@@H](OC(=O)CC(C)C)[C@@H]12]

A method of synthesizing NDM from Neurolenin D is described in Example 2 below.

As used herein, a “therapeutically effective dose” or a “therapeutically effective amount” of a compound, including a crystalline form thereof, or a pharmaceutically acceptable salt thereof, refers to an amount of the compound, or a crystalline form thereof, or a pharmaceutically acceptable salt thereof, which is effective, upon single or multiple dose administration to an organism, in treating a cell, or in curing, alleviating, relieving or improving the organism with a disorder beyond that expected in the absence of such treatment.

As used herein, a “formulation” of a compound is a composition comprising the compound together with one or more pharmaceutically acceptable excipients, suitable for delivery to an organism for the treatment of a disease.

Neurolenin D is a natural product that can be isolated from the perennial flowering plant Neurolaena lobata, commonly known as jackass bitters. N. lobata is widely distributed in Central and South America and is a commonly used plant in Mayan folk medicine. Traditional uses include the treatment of parasitic ailments.

The preparation of NDM from Neurolenin D involves first isolating Neurolenin D from N. lobata, and then mesylating Neurolenin D by reaction with mesyl chloride, according to the scheme:

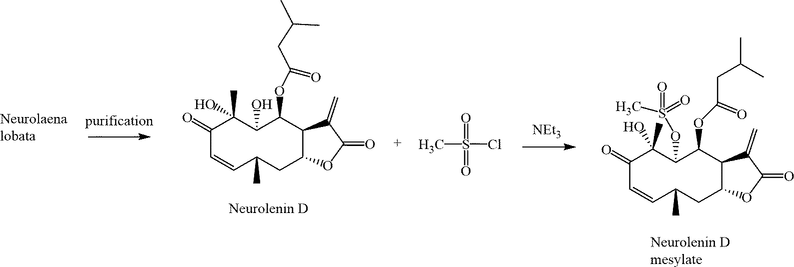

The details of this procedure are provided in Examples 1 to 3 below.

Additional Neurolenin D esters are herein disclosed, having the formulae:

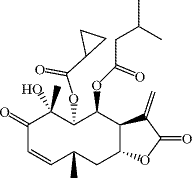 [SMILES: C=C1C(=O)O[C@@H]2C[C@H](C)/C=C\C(=O)[C@](C)(O)[C@@H](OC(=O)C3CC3)[C@@H](OC(=O)CC(C)C)[C@@H]12]

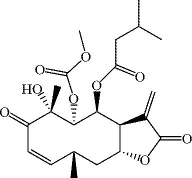 [SMILES: C=C1C(=O)O[C@@H]2C[C@H](C)/C=C\C(=O)[C@](C)(O)[C@@H](OC(=O)OC)[C@@H](OC(=O)CC(C)C)[C@@H]12]

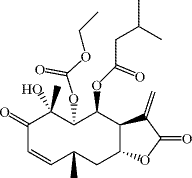 [SMILES: C=C1C(=O)O[C@@H]2C[C@H](C)/C=C\C(=O)[C@](C)(O)[C@@H](OC(=O)OCC)[C@@H](OC(=O)CC(C)C)[C@@H]12]

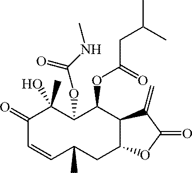 [SMILES: C=C1C(=O)O[C@@H]2C[C@H](C)/C=C\C(=O)[C@](C)(O)[C@@H](OC(=O)NC)[C@@H](OC(=O)CC(C)C)[C@@H]12]

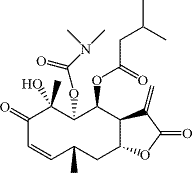 [SMILES: C=C1C(=O)O[C@@H]2C[C@H](C)/C=C\C(=O)[C@](C)(O)[C@@H](OC(=O)N(C)C)[C@@H](OC(=O)CC(C)C)[C@@H]12]

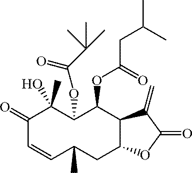 [SMILES: C=C1C(=O)O[C@@H]2C[C@H](C)/C=C\C(=O)[C@](C)(O)[C@@H](OC(=O)C(C)(C)C)[C@@H](OC(=O)CC(C)C)[C@@H]12]

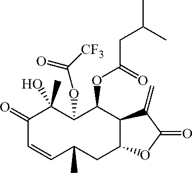 [SMILES: C=C1C(=O)O[C@@H]2C[C@H](C)/C=C\C(=O)[C@](C)(O)[C@@H](OC(=O)C(F)(F)F)[C@@H](OC(=O)CC(C)C)[C@@H]12]

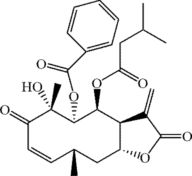 [SMILES: C=C1C(=O)O[C@@H]2C[C@H](C)/C=C\C(=O)[C@](C)(O)[C@@H](OC(=O)c3ccccc3)[C@@H](OC(=O)CC(C)C)[C@@H]12]

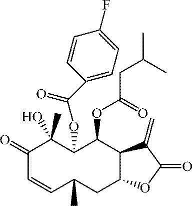 [SMILES: C=C1C(=O)O[C@@H]2C[C@H](C)/C=C\C(=O)[C@](C)(O)[C@@H](OC(=O)c3ccc(F)cc3)[C@@H](OC(=O)CC(C)C)[C@@H]12]

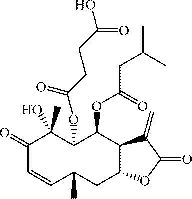 [SMILES: C=C1C(=O)O[C@@H]2C[C@H](C)/C=C\C(=O)[C@](C)(O)[C@@H](OC(=O)CCC(=O)O)[C@@H](OC(=O)CC(C)C)[C@@H]12]

### EXAMPLES

### Example 1: Extracting Neurolenin D From Neurolaena Lobata

### Soxhlet Extraction

Neurolenin D was extracted from the plant Neurolaena lobata by continuous extraction with a Soxhlet extractor. Dried plant leaves of N. lobata from Belize were purchased from Grenada Market in Brooklyn, NY. A bag of N. lobata was ground to powder in a food processor and the powder was added to a cellulose extraction thimble which was filled ⅘ of the way with powder. The thimble was placed into the main chamber of a Soxhlet extractor. To a 1000-mL round bottomed flask, a stir bar and dichloromethane (600 mL) were added. The flask was connected to the main chamber of the Soxhlet extractor. A condenser cooled with a steady flow of cold water was placed atop the main chamber. The dichloromethane was heated to its reflux temperature by a heating mantle connected to a variable voltage outlet. The voltage was set to 10 V and the Soxhlet apparatus was watched carefully until the reflux ring, the highest point of condensation, reached the second bulb of the condenser. If the reflux ring did not reach the second bulb of the condenser, voltage was increased until the ring reached that point. Likewise, the voltage was decreased if the reflux ring reached above the second bulb. Once the reflux ring and temperature remained constant, the extraction was left to run for 24 h.

### Charcoal Filtration

After cooling the extraction, the dichloromethane was rotovapped off to leave behind a green viscous substance in the flask. The green concentrate was dissolved in ethyl acetate (500 mL) and transferred to a 1000-mL Erlenmeyer flask. Three large scoopulas of activated charcoal were added to the Erlenmeyer, and the flask was left to stir for at least 3 hours and up to 24 hours. After the charcoal treatment, the suspension was gravity filtered to remove the charcoal. This entire process was repeated two more times, for a total of three activated charcoal treatments. Once the resulting filtrate was clear in color, the ethyl acetate was rotovapped off the yield a light green viscous oil.

### Column Chromatography

Thin layer chromatography (TLC) confirmed the sample contained a mixture of neurolenin D (R_{f} ~ 0.3), neurolenin C (R_{f} ~ 0.4) and neurolenin B (R_{f} ~ 0.5). The sample was dissolved in the least amount of eluent possible and loaded onto the column, after which flash column chromatography was performed using silica gel (1:100 sample: silica) and eluent (1:1 hexanes: ethyl acetate). The fraction number at which the sample eluted from the column varied widely. However, the pattern of elution was constant: neurolenin B was first to elute, followed by neurolenin C, and then neurolenin D. Fractions containing only neurolenin D were combined and rotovapped to prepare for recrystallization.

### Recrystallization

The least amount of ethyl acetate possible (~5 mL) was added to dissolve the impure neurolenin D obtained from the column. This solution was transferred to a small Erlenmeyer flask with a stir bar and heated carefully on a hotplate. Two Erlenmeyer flasks of heptane (20 mL) and ethyl acetate (10 mL) were also heated to boiling via the hotplate. The boiling heptane was added dropwise to the solution of neurolenin D until the solution turned cloudy, indicating that the product had precipitated out. Once cloudy, a minimal amount of refluxing ethyl acetate was added dropwise until the solution was clear again. The Erlenmeyer was removed from the hotplate, covered with aluminum foil, and left to cool in the freezer for a day or two, resulting in the formation of yellowish white crystals.

### Cleaning Crystals

The crystals were separated from the mother liquor via suction filtration using a Buchner funnel. Small amounts of ice-cold heptane and ethyl acetate were used to rinse the crystals until there were no hints of yellow. The white crystals were left to dry open to the air.

### Example 2: Chemical Synthesis of Neurolenin D Mesylate

The chemical synthesis of NDM proceeds through esterification of neurolenin D with mesyl chloride, according to:

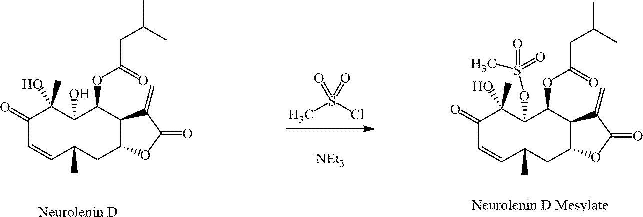

### Procedure

A three-necked round bottomed flask containing neurolenin D (50 mg, 0.13 mmol, 1 equiv) was equipped with a condenser, rubber septum, ground glass stopper, and a stir bar. The flask was flushed with nitrogen and anhydrous dichloromethane (5 mL) was added. The flask was immersed in a dry ice bath at 0° C. Anhydrous triethylamine (40.4 µL, 0.29 mmol, 2.2 equiv) was added, followed by dropwise addition of mesyl chloride (15.1 µL, 0.20 mmol, 1.5 equiv). The resulting mixture was allowed to stir for one hour at 0° C., after which the flask was immersed in an oil bath at 30° C. and left to stir overnight.

### Workup

The reaction was diluted with dichloromethane, quenched with saturated NaHCO₃ (5 mL) and transferred to a separatory funnel. The aqueous layer was extracted with dichloromethane. The combined organic layers were washed with HCl (1M, 3 × 10 mL), saturated NaHCO₃ (2 × 5 mL), and brine (2 × 5 mL). The resulting solution was dried with MgSO₄ and rotovapped to yield a yellowish oil.

### Recrystallization

The yellow oil was dissolved with the least amount of ethyl acetate (~5 mL) and transferred to a small Erlenmeyer flask with a stir bar. The solution, along with two Erlenmeyer flasks of heptane (20 mL) and ethyl acetate (10 mL), were heated to boiling via a hotplate. Boiling heptane (~6 mL) was added dropwise to the solution until it turned cloudy, after which refluxing ethyl acetate was added dropwise until the solution was clear again. The flask was removed from the hotplate, covered with aluminum foil, and allowed to cool in the freezer for a day or two, until white crystals of neurolenin D mesylate (27.0 mg, 0.06 mmol, 45% yield) had formed. If needed, the crystals were cleaned to the point of whiteness following the procedure of cleaning neurolenin D crystals.
¹H NMR (500 MHz, CDCl₃) δ 6.60 (d, 1H), 6.37 (s, 1H), 6.05 (t, 1H), 5.85 (s, 1H), 5.65 (d, 2H), 5.19 (d, 1H), 4.52 (dd, 1H), 4.14 (s, 1H), 3.11 (s, 1H), 3.11 (m, 3H), 2.58 (s, 1H), 2.19 (qd, 2H), 2.00 (m, 1H), 1.85 (td, 1H), 1.57 (d, 8H), 1.45 (td, 1H), 1.16 (d, 3H), 0.90 (d, 7H) ppm
¹³C NMR (125 MHz, CDCl₃) δ 204.0, 172.0, 168.9, 149.0, 135.0, 127.0, 125.2, 81.7, 79.9, 77.3, 73.2, 43.2, 41.3, 40.3, 38.8, 28.5, 25.3, 22.5, 19.9, 0.3 ppm
HRMS (ESI): calculated for C₂₁H₃₀O₉S [M + Na] 481.5130, found 481.1491

### Example 3: Biological Properties of Neurolenin D Mesylate (NDM)

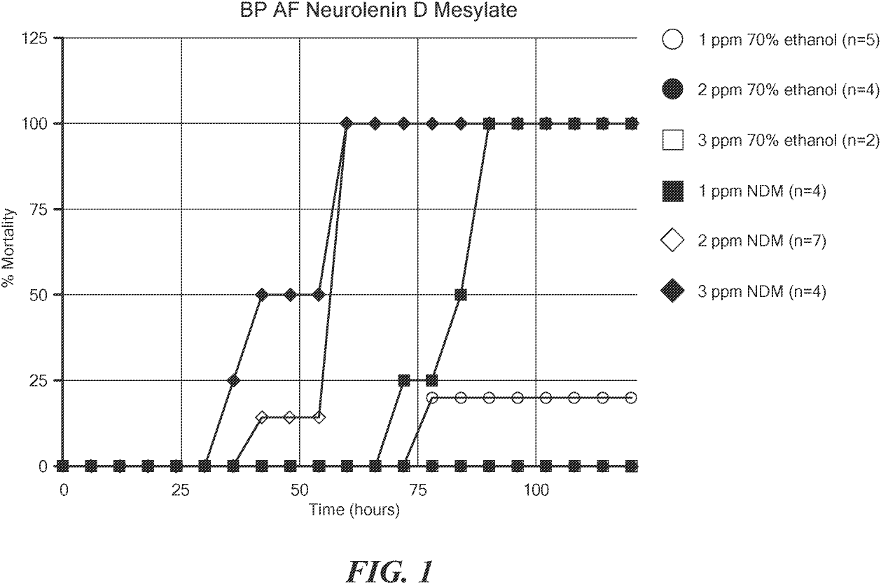

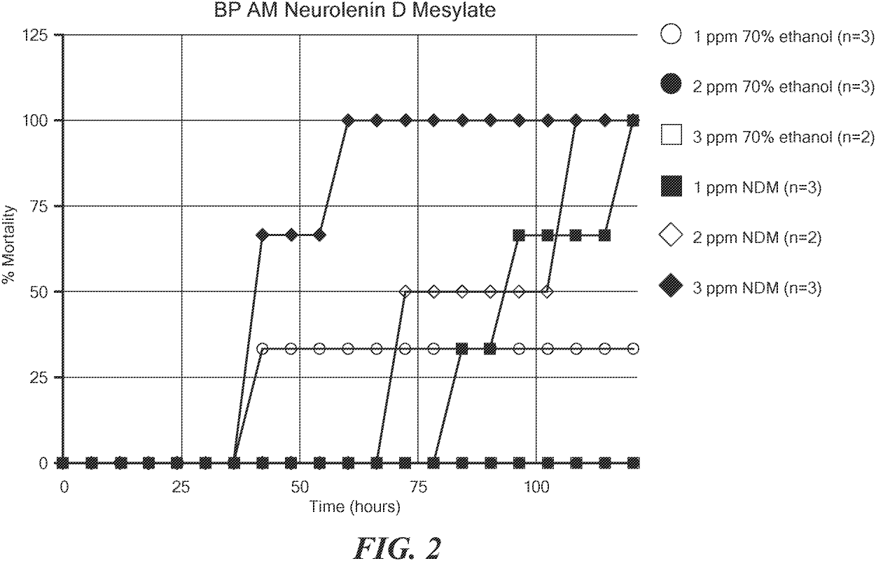

As demonstrated in FIGS. 1 and 2, Neurolenin D mesylate shows remarkable potency in killing both male and female adults of the parasitic nematode Brugia pahangi (BP) in culture. BP infects a variety of animals and occasionally humans and is very closely related to the important human parasite Brugia malayi (BM), which is one of the major causative agents of lymphatic filariasis and elephantiasis in humans. Because biologically and biochemically, BP is very similar to the human parasite BM, it is anticipated that NDM will show similar potency in killing this devastating human parasite. Moreover, because previous drugs developed for treating nematode parasite infections have worked to kill a wide variety of parasite species, it is further anticipated that neurolenin D mesylate will have similar broad spectrum effectiveness on human, animal and plant nematode parasites.

Preliminary data on NDM indicates that the compound is not toxic or mutagenic in biological activity assays.

### NDM Kills Adult Female BP Parasites in Culture

FIG. 1 shows data demonstrating the ability of neurolenin D mesylate (NDM) to kill adult female BP parasites (BP AF) in culture. The drug was applied at 1, 2 and 3 parts per million (ppm) and was 100% effective at killing the parasites at all three concentrations. Even at 1 ppm, all parasites were killed within 90 hours after treatment. The control treatment is with 1, 2 and 3 ppm ethanol given to the parasites in culture under the same conditions. One adult female parasite treated with 1 ppm ethanol died at about 80 hours. This death is not experimentally significant as occasionally a parasite dies in culture without any drug treatment.

### NDM Kills Adult Male BP Parasites in Culture

FIG. 2 shows data demonstrating the ability of neurolenin D mesylate (NDM) to kill adult male BP parasites (BP AM) in culture. The drug was applied at 1, 2 and 3 parts per million (ppm) and was 100% effective at killing the parasites at all three concentrations. Even at 1 ppm, all parasites were killed within 120 hours after treatment. The control treatment is with 1, 2 and 3 ppm ethanol given to the parasites in culture under the same conditions. One adult male parasite treated with 1 ppm ethanol died at about 40 hours. This death is not experimentally significant as occasionally a parasite dies in culture without any drug treatment.

### NDM Kills Parasitic Nematodes in Adult and Larval Forms

As shown in FIGS. 1 and 2, NDM is very effective at killing adult male and adult female BP parasites. These data have been repeated in many separate trials with the same results seen. 100% of all adult male and female parasites are always killed upon treatment with NDM. In repeated experiments we have also demonstrated that NDM kills 100% of the microfilariae (larvae) that are produced following the mating of adult male and female parasites.

This combined mode of action of killing both larval and adult forms is important since killing microfilariae is important for preventing transmission of the disease, while killing adult worms is important for eliminating the infection in the diseased individual (and ultimately preventing transmission).

When the results shown in FIGS. 1 and 2 are compared with the results of other neurolenin derivatives, the parasites are killed more slowly with NDM than with other neurolenin derivatives, even though at the endpoint, NDM kills 100% of the worms. This surprising result is promising since killing the parasites too rapidly can result in a severe immunological reaction, in some cases causing death of the patient (Loa loa).

Moreover, because all nematodes share similar biology and biochemistry, it is very likely that this new drug will be effective in killing a wide variety of nematode parasites that infect 1.5 billion people worldwide and are important drivers of the cycle of disease and poverty in low and middle income countries. It should be noted that these diseases are not solely limited to the low and middle income countries. One of these diseases, hookworm, is still found in the United States for example. In addition to human diseases, nematode parasites are important agents of disease in companion animals and livestock. Examples include dog heartworm disease and roundworm diseases of cattle.

Parasitic nematodes are also important pathogens of economically important crops and include root knot nematode and many others nematode species. Together these diseases infect billions of humans and have an incredible economic impact in terms of infections of companion animals, livestock and crop plants. Because the drugs used to treat these diseases have been used routinely for many years and have been overused, drug resistance is a well-documented problem in some species and drives the need for new and potent drugs to kill these common pathogens. NDM is a new compound that could make an important contribution to controlling these pathogens.

### Example 4: Chemical Synthesis of Additional Neurolenin D Esters

Further Neurolenin D esters can be synthesized by reaction of neurolenin D with an acyl chloride or with an anhydride. For the acyl chloride reaction the scheme is:

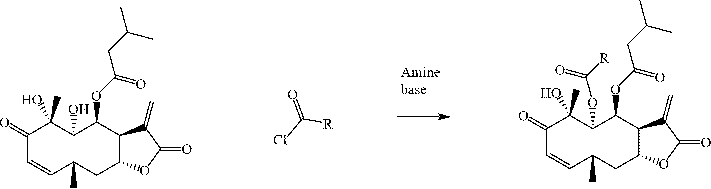

Whereas for the anhydride reaction the scheme is:

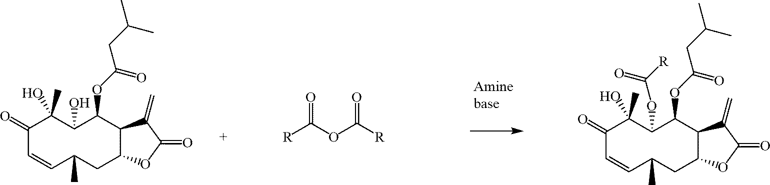

For either reaction scheme, R is a moiety selected from the group consisting of:

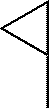

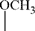

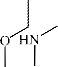

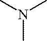

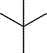

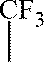

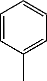

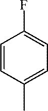

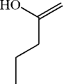

In some embodiments, the amine base is trimethylamine (NEt₃). In some embodiments, the amine base is selected from the group consisting of pyridine, Diazabicycloundecene (DBU), imidazole, 4-Dimethylaminopyridine (DMAP), and 1,4-Diazabicyclo[2.2.2]octane (DABCO).

The embodiments of the invention described above are intended to be merely exemplary; numerous variations and modifications will be apparent to those skilled in the art. All such variations and modifications are intended to be within the scope of the present invention as defined in any appended claims.

What is claimed is:

1. A compound having the formula: 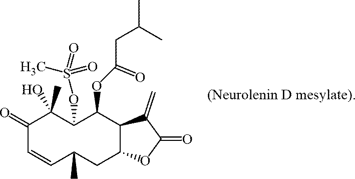
.

2. A formulation comprising neurolenin D mesylate for the treatment of an organism suffering from a disease caused by an infection with a parasitic nematode, the organism selected from the group consisting of human, animal, and plant.

3. A method of synthesizing neurolenin D mesylate comprising:
reacting neurolenin D with mesyl chloride to form Neurolenin D mesylate.

4. A method of treating an organism suffering from a disease caused by an infection with a parasitic nematode, the organism selected from the group consisting of human, animal, and plant, the method comprising administering to the organism a therapeutically effective dose of neurolenin D mesylate.

5. The method of claim 4 wherein the organism is a human.

6. The method of claim 4 wherein the organism is a companion animal.

7. The method of claim 4 wherein the organism is livestock.

8. The method of claim 4 wherein the organism is a plant.

9. The method of claim 4 wherein the parasitic nematode is a causative agent of a disease selected from the group consisting of lymphatic filariasis, elephantiasis, and river-blindness in humans.

10. The method of claim 4 wherein the parasitic nematode is a causative agent of heartworm in dogs.

11. The method of claim 4 wherein the parasitic nematode is a causative agent of a roundworm disease in livestock.

12. A compound selected from the group consisting of: 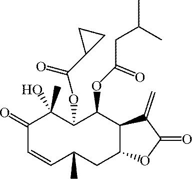 [SMILES: C=C1C(=O)O[C@@H]2C[C@H](C)/C=C\C(=O)[C@](C)(O)[C@@H](OC(=O)C3CC3)[C@@H](OC(=O)CC(C)C)[C@@H]12] 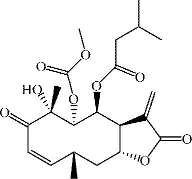 [SMILES: C=C1C(=O)O[C@@H]2C[C@H](C)/C=C\C(=O)[C@](C)(O)[C@@H](OC(=O)OC)[C@@H](OC(=O)CC(C)C)[C@@H]12] 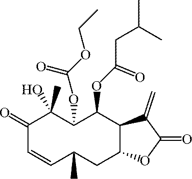 [SMILES: C=C1C(=O)O[C@@H]2C[C@H](C)/C=C\C(=O)[C@](C)(O)[C@@H](OC(=O)OCC)[C@@H](OC(=O)CC(C)C)[C@@H]12] 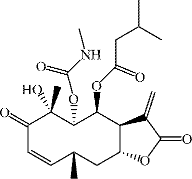 [SMILES: C=C1C(=O)O[C@@H]2C[C@H](C)/C=C\C(=O)[C@](C)(O)[C@@H](OC(=O)NC)[C@@H](OC(=O)CC(C)C)[C@@H]12] 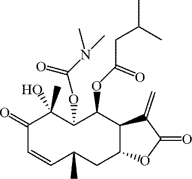 [SMILES: C=C1C(=O)O[C@@H]2C[C@H](C)/C=C\C(=O)[C@](C)(O)[C@@H](OC(=O)N(C)C)[C@@H](OC(=O)CC(C)C)[C@@H]12] 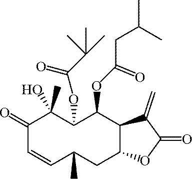 [SMILES: C=C1C(=O)O[C@@H]2C[C@H](C)/C=C\C(=O)[C@](C)(O)[C@@H](OC(=O)C(C)(C)C)[C@@H](OC(=O)CC(C)C)[C@@H]12] 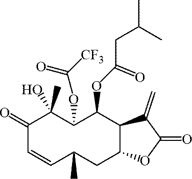 [SMILES: C=C1C(=O)O[C@@H]2C[C@H](C)/C=C\C(=O)[C@](C)(O)[C@@H](OC(=O)C(F)(F)F)[C@@H](OC(=O)CC(C)C)[C@@H]12] 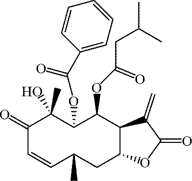 [SMILES: C=C1C(=O)O[C@@H]2C[C@H](C)/C=C\C(=O)[C@](C)(O)[C@@H](OC(=O)c3ccccc3)[C@@H](OC(=O)CC(C)C)[C@@H]12] 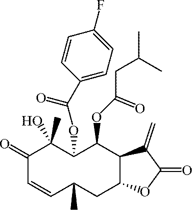 [SMILES: C=C1C(=O)O[C@@H]2C[C@H](C)/C=C\C(=O)[C@](C)(O)[C@@H](OC(=O)c3ccc(F)cc3)[C@@H](OC(=O)CC(C)C)[C@@H]12] 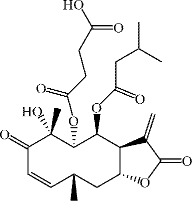 [SMILES: C=C1C(=O)O[C@@H]2C[C@H](C)/C=C\C(=O)[C@](C)(O)[C@@H](OC(=O)CCC(=O)O)[C@@H](OC(=O)CC(C)C)[C@@H]12]
.

13. A formulation comprising one or more of the compounds of claim 12 for the treatment of an organism suffering from a disease caused by a parasitic nematode, the organism selected from the group consisting of human, animal, and plant.

14. A method of synthesizing neurolenin D esters comprising:
reacting neurolenin D with a compound having a formula selected from the group consisting of 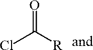 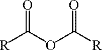
wherein the reaction conditions include the presence of an amine base, and wherein R is selected from the group consisting of: 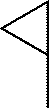 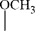 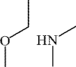 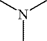 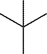 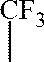 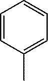 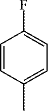 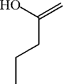
.

15. The method according to claim 14 wherein the compound reacting with neurolenin D has the formula 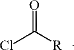
.

16. The method according to claim 14, wherein the compound reacting with neurolenin D has the formula 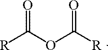
.

17. The method according to claim 14, wherein the amine base is selected from the group consisting of pyridine, Diazabicycloundecene (DBU), imidazole, 4-Dimethylaminopyridine (DMAP), and 1,4-Diazabicyclo[2.2.2]octane (DABCO).

18. A method of treating an organism suffering from a disease caused by an infection with a parasitic nematode, the organism selected from the group consisting of human, animal, and plant, the method comprising administering a therapeutically effective dose of one or more of the compounds of claim 12.

19. The method of claim 18 wherein the organism is a human.

20. The method of claim 18 wherein the organism is a companion animal.

21. The method of claim 18 wherein the organism is livestock.

22. The method of claim 18 wherein the organism is a plant.

23. The method of claim 18 wherein the parasitic nematode is a causative agent of a disease selected from the group consisting of lymphatic filariasis, elephantiasis, and river-blindness in humans.

24. The method of claim 18 wherein the parasitic nematode is a causative agent of heartworm in dogs.

25. The method of claim 18 wherein the parasitic nematode is a causative agent of a roundworm disease in livestock.
